(19) United States
(12) Patent Application Publication
(10) Pub. No.: US 2009/0124520 A1
(43) Pub. Date: May 14, 2009

(54) NOVEL HYDRATE BASED SYSTEMS

(75) Inventor: Bahman Tohidi, Midlothian (GB)

Correspondence Address: YOUNG & BASILE, P.C., 3001 WEST BIG BEAVER ROAD, SUITE 624, TROY, MI, 48084, US

(73) Assignee: Heriot-Watt University, Edinburgh (GB)

(21) Appl. No.: 11/718,654

(22) PCT Filed: Nov. 3, 2005
(86) PCT No.: PCT/GB2005/004267

§ 371 Date: Dec. 5, 2008

(30) Foreign Application Priority Data
Nov. 4, 2004 (GB) 0424387.9

(51) Int. Cl. C09K 8/52; F25B 13/00; F16L 3/00; F17D 1/00; C07C 9/00

(52) U.S. Cl. 507/90; 62/324.1; 137/561 R; 137/1; 585/15

(57) ABSTRACT

A method for transporting a fluid comprising a clathrate forming gas through a transportation system including a pipeline. The method involves subjecting the fluid to clathrate forming temperature and pressure conditions and introducing sufficient of a clathrate forming host to convert substantially all of the gas to clathrate and to form a flowable slurry. The flowable slurry is then conveyed through a pipeline to a destination. An alternative method involves transporting the fluid by means of a ring pipeline containing a carrier fluid, which includes clathrate inhibitors. Also provided is a heat pump whose working fluid is a clathrate forming composition.

### RELATED INVENTION

This application claims priority to PCT Application No. PCT/GB2005/004267 filed on Nov. 3, 2005.

### FIELD OF THE INVENTION

The invention described herein belongs particularly to the fields of petroleum engineering, oil and gas transportation and deepwater development, but has a range of applications outside of these fields.

### BACKGROUND TO THE INVENTION

Gas hydrates are crystalline compounds formed as a result of a physical combination of water and suitably sized molecules, for example, C₁, C₂, C₃ hydrocarbons, or various combinations of the above. Other compositions comprising suitable ‘host’ substances other than water combined together with suitable ‘guest’ substances are known. Such guest/host compositions, including the gas hydrates mentioned above, are generally known as “clathrates”.

They resemble ice, but unlike ice they can form at temperatures well above the freezing point.

Preventing problems stemming from gas hydrate formation is a major flow assurance challenge in oil and gas production and transportation.

There are several options available to reduce the risks associated with gas hydrate formation, including in particular dehydration (the removal of water from a system), injection of thermodynamic and/or low dosage hydrate inhibitors (kinetic inhibitors and anti-agglomerates), and insulation of the system or active heating, in order to keep system operating conditions outside the hydrate stability zone.

The problem is that all of these options are expensive. Furthermore, in some cases there is no current solution, for example in deepwater or long tieback operations.

Industry has always seen water as the source of the problem in gas hydrate formation, and so has always tried to remove or reduce the aqueous phase (for example by using downhole and/or subsea water separators).

All the existing hydrate flow assurance concepts are based on reducing or removing water, or preventing or delaying hydrate formation.

Recently, however, a new terminology entitled ‘Cold Flow’ has been introduced where the objective is to be able to transport oil or gas without any thermal treatment, such as insulation or active heating, reducing the costs of deepwater pipeline installation.

The ‘Cold Flow’ process is described in U.S. Pat. No. 6,774,276. In the described process gas hydrates are deliberately formed in hydrocarbon fluids to convert the water (liquid or vapour) present in the fluid to a solid, according to a particular process. The water present in the hydrocarbon fluids, (which are typically oil/gas/water mixtures), is thereby removed or locked up as a solid hydrate. British Petroleum has been supporting a project by SINTEF in Norway, exploring this technology. Their idea is based on recycling part of the hydrates and oil present in the system back upstream using a pump and recirculating loop, as oil is the continuous phase. The idea is that the recycled hydrate particles provide nucleation sites, and the cooled fluid (oil) a heat sink, to facilitate non-sticky (dry) hydrate formation. It is argued that hydrates which form from inside to outside (i.e. about a pre-formed hydrate particle) are dry and do not stick to each other, and hence are capable of flowing without creating the blockage problems usually associated with gas hydrate formation. These dry hydrates are claimed to be transportable in a continuous oil phase. However, from a practical viewpoint there are many difficulties, from the design of the recycling systems and the amount of recycling fluids necessary, to coping with changes in the fluid systems and pipeline throughput, along with dealing with start-up and shut-downs. Furthermore, from the description available in the literature it seems that the system is limited to oil/condensate systems with relatively low water cuts and low gas to oil ratios, and probably not applicable to saline systems, as it won't be possible to convert all the water into hydrates, i.e., form dry hydrates. The work is also discussed at http://www.ntnu.no/gemini/2003-06e/28-31.htm and http://remotemonitoringconference.com/pdflsession2_{—}1.pdf).

Alternative approaches to avoiding hydrate problems include the use of anti-agglomerate (AA) chemicals. Anti-agglomerate chemicals are used to prevent hydrate particles that do form collecting into larger particles or even forming a solid plug which can block a pipeline. Anti-Agglomerates are not new to the industry. They have been detailed in numerous publications. The anti-agglomerate method was begun by Behar et al. [Behar, E., Sugier, A., Rojey, A., “Hydrate Formation and Inhibition in Multiphase Flow”, presented at BHRA Conference Operation Consequences of Hydrate Formation and Inhibition Offshore, Cranfield UK, Nov. 3, 1988.] and Frostman L. M. [(2000), “Anti-Agglomerate Hydrate Inhibitors for prevention of Hydrate plugs in deepwater systems”, Annual Technical Conference and exhibition in Dallas, Tex., 1-4 Oct., Frostman L. M., Przybylinski J. L., (2001), “Successful applications of Anti-Agglomerate Hydrate Inhibitor”, SPE International Symposium on Oilfield Chemistry, Houston, Tex., 13-16 February].

AAs have been used for preventing gas hydrate formation, but only in systems containing a liquid hydrocarbon phase (generally more than 40%). There is a need for the presence of a liquid hydrocarbon phase in order to utilise the existing AAs, because their mechanism for prevention of blockage caused by hydrate revolves around dispersing water.

As a result, AAs are not currently used in dry natural gas systems or systems containing small amounts of the hydrocarbon phase. This is probably due to the low water-cut in gaseous systems, hence making thermodynamic and/or kinetic hydrate inhibitors the more cost effective option. Furthermore, the existing Anti-Agglomerates probably would not work in systems where there is limited hydrocarbon phase and water is the limiting reactant.

A further problem associated with flow and transport of hydrocarbon fluids is the phenomenon known as ‘slugging’. In the transport of a multiphase fluid, such as an oil/gas/water mixture, separated flow can occur, i.e. slugs of the liquid phase(s) can form in the pipe separated by pockets of gas. This unstable and intermittent flow presents many hazards and can impact seriously on the economics of a hydrocarbon producing system. For example, the gas phase behind a liquid slug becomes compressed because the transportation of a liquid slug requires a larger pressure behind the slug to keep it moving. The arrival of this compressed gas at the outlet of a pipeline or production platform creates a large gas surge threatening the reliable and safe operation of the processing equipment.

Another major problem in oil and gas transport is preventing gas hydrate problems during shut-ins and start-ups. In many cases the main risk of gas hydrate blockage occurs during shut-ins when the pipeline temperatures drop to very low temperatures and during start-up when the system is pressurised to start the fluid flow.

It is an object of the present invention to provide methods for transporting a fluid comprising a gas that avoid or reduces at least some of the aforementioned problems.

It is a further object of the present invention to provide novel uses for clathrates.

It is a further object of the invention to prevent or minimise the problems of gas hydrates associated during shut-ins and/or start-ups by forming a stable hydrate into oil or hydrates in water slurry minimising the risks associated with hydrate blockages.

### DESCRIPTION OF THE INVENTION

According to a first aspect the present invention provides a method for transporting a fluid comprising a clathrate forming gas/compound through a transportation system including a pipeline, said method comprising the steps of:

a) subjecting the fluid to clathrate forming temperature and pressure conditions;

b) introducing sufficient of a clathrate forming host to convert substantially all of the gas/compound to clathrate and to form a flowable slurry; and,

c) conveying the resultant flowable slurry through the transport system to a destination.

It will be understood that the first two steps of the method can be undertaken in any convenient order or even simultaneously, depending on the transportation system and fluid being subjected to the method. For example, where the pipeline of the transportation system is a typical subsea pipeline, in use for hydrocarbon transport, the clathrate will be a hydrate. Hydrate forming temperature and pressure conditions may normally exist in the pipeline. The water may be added (if necessary, as the main objective is to convert most or all of the gas phase into hydrates and/or forming a transportable slurry) to the flow of hydrocarbons before it enters the cooled, pressurised, environment of the pipeline or alternatively, after it is in the pipeline. Other fluids, such as liquid hydrocarbon might be added to the system, especially in gas and/or high gas-to-oil ratio (GOR) systems, for reducing the viscosity of the hydrate slurry with or without AA.

The skilled addressee will readily know how to apply appropriate temperature and pressure conditions to cause hydrate formation, but suitable conditions/processes are described, for example, in U.S. Pat. No. 6,774,276 to which the reader is directed.

By converting all or most of the gas in a hydrocarbon fluid system into a hydrate a number of benefits ensue. The volume of the gaseous component of the hydrocarbon fluid is greatly reduced. As substantially no free gas remains in the system, problems associated with unplanned hydrate formation during transport of the hydrocarbon fluid, especially along a pipeline, is all but eliminated.

The possibility of slugging in multiphase (gas/liquid) flow is also removed or greatly reduced. Significant CAPEX is currently invested in slug catchers and other means of reducing the instability due to slugging in current hydrocarbon production systems. As a result of reducing/eliminating the gas phase, the densities of the various phases remaining are very similar, resulting in a reduction in slugging problems.

For transport of hydrocarbon fluids, the flowable slurry formed may be of a hydrate dispersed in a liquid which is substantially hydrocarbon in nature. Alternatively it may be a hydrate dispersed in a liquid which is substantially water. In some cases the liquid will be a mixture which contains significant amounts of both water and hydrocarbons.

The slurry type can be chosen depending on the composition of the hydrocarbon containing fluid being transported. Where a mixture of hydrocarbons comprises a substantial portion of liquid hydrocarbons and a relatively small amount of gas, then typically only sufficient water to convert all or most of the gas to hydrate will be added, as the hydrate can be slurried in the hydrocarbon liquid (oil) phase. However, more water can be added if desired or required to improve flowability. On the other hand, where the hydrocarbon fluid has a large proportion of gas relative to liquid, sufficient excess water is added to ensure that the hydrate particles are dispersed in sufficient liquid water to form the flowable slurry. Typically a slurry may comprise up to 10%, 20%, or 30% v/v of particles in the slurry. Desirably clathrate agglomeration and system blockage is prevented by the use of suitable anti-agglomerates (AAs). However, it is also possible to add liquid hydrocarbon to the system to improve the transportability of the hydrate slurry with or without AAs. For example, the added clathrate host, for example, water, with or without liquid hydrocarbon could contain the anti-agglomerates as additives. Other additives, to promote clathrate formation and/or modify their crystallisation characteristics can also be added if desired. Conveniently these can also be added with or in the added host, especially if the host is water. For example, in oil and gas transportation applications possible methods of introducing AA in practice include firstly a method whereby water containing AAs and other additives is added into the ‘upstream’ area of the system, well before the receiving platform or destination, and secondly a method whereby water containing AAs and other additives is recycled within the system, which is preferably in the form of a loop. The latter option is the preferred option as it will allow for minimising the usage of chemicals and a lower degree of subcooling, due to the presence of hydrate structures/particles in the circulating water. Suitable Anti-Agglomerates include highly branched quaternised alkyl ammonium or phosphonium compounds (usually with accompanying bromide/chloride ions), as discussed by Klomp et al in U.S. Pat. No. 5,460,728.

The resultant mixture of hydrates, fluids and any residual gas (the ‘hydrocarbon fluid’) then flows through the system.

Although the viscosity within the system is an issue, and lower viscosity is desirable, this method can be performed on any system by adjusting the amount of water, liquid hydrocarbon or other clathrate host in the system to produce the desired result in terms of producing a suitably flowable slurry.

The minimum amount of water required to be added to the system depends on the gas within the system and the system's temperature and pressure conditions, and consequently on the hydrate structure formed and hydration number, and can be expressed as follows:

G.n_{w}(H₂O), where G is a mole of gas and n_{w} is the hydration number

The amount of water added should be higher than n_{w} moles of gas, to assure maximum conversion of gas into hydrate and the formation of hydrate slurry with good transportability. Usually 1-5 vol % of water phase of AA is used. It may be suitable to consider adding liquid hydrocarbons to the system for improving the transportability of hydrate slurry in the presence and absence of AAs.

There are two opposing factors here. Increasing the amount of water (and liquid hydrocarbon, if necessary in high Gas Liquid Ratio systems) will increase the total pipeline flow rate but will result in a low viscosity hydrate slurry, whereas, reducing the amount of water will have a positive impact on the total flow rate of the pipeline but will mean the hydrate content of the slurry is high hence high viscosity. The optimum range of water (and liquid hydrocarbon, if necessary) can and should therefore be determined by experimental means and/or an integrated experimental and modelling approach. The tests conducted in this laboratory show that there could be a reduction in the apparent viscosity of the system as a result of hydrate formation.

This first aspect of the present invention can increase system capacity, for example a single unit by volume of hydrate can accommodate up to 175 units by volume of gas at standard conditions (i.e., hydrate formation is roughly equivalent to 2250 psia pressure), whilst reducing system operating pressure, and hence the cost of construction and operation of the system, as there is no need for system insulation or heating.

It is also possible to regenerate/recover all or part of the AA utilized and recycle them into the system, hence reducing chemical costs and alleviating potential environmental concerns. In the receiving platform, at the system destination, or in the “downstream” area of the system the hydrates are separated from the water/liquid hydrocarbon phase. Various techniques can be used here which are not the objective of this invention. One option is to have a separator where the density difference between hydrates and water is used for their separation. It is also possible to introduce the incoming fluid to the top of a sieve tray. Solid hydrates will remain at the top of the sieve tray whilst liquid water will pass through and will be collected at the base of the separator. The collected water (part or all) and/or the water (part or all) resulted from hydrate dissociation could be recycled. The recycled stream may also contain liquid hydrocarbons to improve the transportability of the hydrate crystals. Other separation techniques are also possible, for example in the case of ionic AAs, the application of ion exchange units, and for polymeric AAs, membrane filtration units at destination may be considered. It should also be noted that part or all of the circulating water can come from the dissociated hydrates unless it is decided to transfer all hydrates contained within the system as solids or hydrate in oil or hydrate in water slurries.

Another potential benefit can come from a reduction in overall system pressure, due to lower flow velocities and pressure recovery. In single or multi-phase flow the pressure within the system is reduced during uphill movement of the fluid(s). It is recovered (increased) in single-phase flow during downhill movement. In the case of gas-liquid flow pressure is not recovered in downhill movement as the gas phase is not compressed. Therefore, in single-phase flow the hydrostatic pressure drop depends on the difference between inlet and outlet elevations, but in gas-liquid flow the hydrostatic pressure drop is the summation of pressure changes caused by all uphills. Furthermore, in gas-liquid fluid flow the frictional pressure drop depends on the flow regime and the superficial gas and liquid velocities, which could be higher than single phase pressure drops. Finally, there is an element of acceleration pressure drop in systems containing gas, which is negligible in incompressible fluid flow. Flow velocity will depend on the density of the fluids and the total mass flow rate. While the density of the hydrate slurry of the present invention is much higher than gas-liquid systems, the total mass flowrate could be higher as water is deliberately added to the system. As a result the velocity depends on the amount of water added to the system, as discussed earlier when relating the amount of water to the system viscosity.

This potential benefit is supported by experimental data examining the effect of hydrocarbon hydrate formation on the transportability of the fluid (by measuring the torque applied to a mixer in a test rig) and calculations relating the hydrate slurry concentration to the pressure drop. The results showed that the flow of a hydrate forming gas/liquid as a hydrate slurry has a reduced pressure drop in comparison to the two phase water-oil flow.

Furthermore, as hydrate formation is an exothermic process, the heat generated during hydrate formation can be used to maintain a beneficial system temperature, particularly reducing the risks associated with wax formation, as discussed by Misra S, Baruah S, Singh K [“Paraffin problems in crude oil production and transportation—A Review”, SPE Production & Facilities, 10 (1): 50-54 FEB 1995], Nenniger, J. E., Cutten, F. B., Shields, S. N. [‘Wax Deposition in a WAG Flood’, SPE 14688] and Newberry, M. E. [‘Crude Oil Production and Flowline Pressure Problems’, SPE 11561].

Finally, by recycling a percentage of the fluid phase which contains the chemicals (AAs, corrosion and scale inhibitors and other additives and such like), reducing the operational costs, the fluid acts as a carrier bringing the hydrocarbon fluids from their source to destination.

The hydrates transferred upstream to the receiving platform, surface facilities, or system destination could then either be transported as solid hydrates (dry or hydrate in oil slurry or hydrate in water slurry), or dissociated by supplying heat and/or depressurisation.

The heat source for dissociation could be seawater or air, which will result in a reduction in their temperatures. This cooling effect, derived from the enthalpy of dissociation of the clathrate (hydrate) can be utilised. In the case of air, the resulting cold air could be used for air conditioning purposes and/or producing fresh water, as the equilibrium concentration of water in the air reduces with a reduction in the system temperature, hence the extra water vapour will condense as fresh water.

According to a second aspect the present invention provides a method for transporting a fluid comprising a clathrate forming gas/liquid as described above where the transportation system comprises a ring pipeline. In a ring pipeline or ‘ring main’ system a circulating carrier fluid/fluids (in the case of including liquid hydrocarbon in the system to improve the transportability of hydrates and/or performance of AAs in highly gaseous systems) flows/flow round the ring pipeline. The carried fluid (e.g., hydrocarbon reservoir/well stream(s)) comprising a clathrate forming gas, which is to be transported in the form of a flowable clathrate slurry, is inserted/introduced by means of a suitable inlet system into the ring pipeline from a source or sources. The circulating carrier fluid then transports the fluid to a destination where it is partly or fully abstracted from the carrier fluid.

Conveniently the circulating carrier fluid is, for example, water or water+liquid hydrocarbon+other fluids (for improving the transportability of the hydrate slurries).

For example, individual wells or other sources of fluid hydrocarbons are each connected to this ring pipeline with appropriate flow control or choke systems. The circulating fluid, which may be water, hydrocarbon or a mixture of both, acts as a carrier, transferring hydrocarbon fluids from individual wells to the production facilities. The production facilities may be at one destination (location) or more than one in a more complex system. Again the amount of clathrate host is adjusted to convert all or most of the gas phase into clathrate. This conversion can be carried out as the fluid is transported through the pipeline or before, depending on circumstances. If necessary, the ring pipeline diameter can be gradually increased, to accommodate the extra fluid, as it passes and collects from more sources of hydrocarbon fluid (wells) on its way to a destination. The hydrate slurry is transported with the carrier fluid to the platform or processing unit where hydrates and hydrocarbon liquid can be separated from the carrier fluid. Anti-agglomerates and other additives might be necessary to prevent flow blockage in this system as for in a conventional pipeline arrangement. Some or all of the water separated and/or the water resulting from hydrate dissociation can be circulated to minimise inhibitor consumption (depending on the various inhibitors distributions and/or other operational circumstances). It might be necessary to add make up water with or without inhibitors in order to achieve the desired hydrate slurry mixtures.

A ring pipeline with a circulating carrier fluid can provide a particularly economic way of carrying out the method of the invention, especially when a number of production wells are feeding a production facility, with efficient reuse of carrier fluid. It is also possible to increase the amount of carrier fluid gradually (e.g., additional connections to the ring pipeline) prior or after introduction of a new well to the ring pipeline.

According to a third aspect the present invention provides a transport system for a fluid comprising a clathrate forming gas/liquid, said system comprising a ring pipeline having a circulating carrier fluid (or fluids) comprising clathrate-inhibiting compounds. As with the second aspect of the invention, in this aspect the ring pipeline can connect a number of fluid sources (for example oil wells) to one or more destinations (production facilities). In this aspect of the invention, as applied to hydrocarbon transport where unplanned hydrate formation is a danger, circulating the carrier fluid together with thermodynamic and/or kinetic hydrate inhibitors prevents formation of gas hydrate. As gas hydrate formation is prevented, multiphase (liquid and gas) flow is not excluded by this arrangement, unlike in the other aspects of the invention described above. Nevertheless, use of a ring pipeline allows the circulating carrier fluid and its associated hydrate inhibitor compounds to be easily and continuously recycled. Preferably the circulating carrier fluid comprises water. Water is readily separable from hydrocarbons.

The present invention also provides a method for transporting a flow of fluid comprising a gas said method comprising the steps of: inserting said fluid via an inlet into a ring pipeline, said ring pipeline having a circulating carrier fluid comprising clathrate-inhibiting compounds; and abstracting said fluid from said circulating carrier fluid at a destination. Preferably the circulating carrier fluid comprises water.

A fourth aspect of the present invention is based on artificially forming and dissociating clathrates and employing their heat source and heat sink characteristics.

According to a fourth aspect the invention provides a heat pump wherein the working fluid comprises a clathrate forming host and a clathrate forming guest said host and said guest forming a clathrate and then dissociating back to the host and the guest under the influence of pressure changes. The clathrate may be, for example a hydrate, where the host comprises water. In the description that follows it will be understood that the expressions ‘hydrate’ and ‘hydrates’ refer to any suitable clathrate as well as to clathrates where water is actually the host.

The invention is based on a method whereby water, or another hydrate forming host is mixed with one or several clathrate or hydrate forming compounds to produce a single or near single-phase liquid phase within a system (with or without using chemicals). This working fluid can then be used in a heat pump. This system is preferably a closed vertical or near vertical loop, preferably in a media where the lower part of the loop is exposed to low temperature fluid (for example within an ocean environment). A system whereby changes in system pressure and temperature similar to those present within a vertical or near vertical system due to height can be artificially implemented can also be employed within this aspect of the present invention. The hydrate forming mixture can be circulated with the help of a pump to regulate the flow rate and the timing. Hydrates are formed at the lower pressurised part of the loop due to an increase in the system pressure and preferably a reduction in the system temperature. This reaction will result in the release of heat. The hydrates are dissociated at the upper part of the loop due to a reduction in the system pressure and potentially an increase in the surrounding temperature. This reaction will result in absorbing heat from the surroundings. The resulting system works as a hydrate heat pump or cycle, which can consequently be used in many applications including air conditioning, producing fresh water and transferring heat to the seabed. It is also possible to switch the hydrate forming and dissociation sections by changing the hydrate forming (guest) compounds. By changing the guest compound it would be possible to dissociate hydrates as a result of an increase in the pressure and form hydrates as a result of reducing pressure.

With regard to the fourth aspect of the present invention it is important to remember that hydrates are solid compounds formed as a result of combinations of suitably sized molecules with hydrate forming fluid compounds under low temperature and high pressure conditions, and that their formation is an exothermic process and their dissociation an endothermic process. A reduction in temperature generally promotes hydrate formation whereas the effect of pressure could depend on the characteristics of the guest molecule.

This hydrate pump/cycle is preferably composed of a closed loop, circulating a hydrate forming fluid/gas mixture through a ‘downward’ leg, where the system is pressurised, and a ‘return’ leg, where the system becomes depressurised.

This causes the system to work as a heat source under pressurised conditions, and a heat sink when depressurised, through hydrate formation and dissociation, respectively.

Hydrates form as a result of an increase in the system pressure and a reduction in the system temperature on the downward leg as the hydrostatic pressure increases.

As hydrate formation is an exothermic process, i.e. increasing the system temperature, it results in a heat flow from the system to the surrounding environment. This heat energy can serve to provide heat to subsea installations.

The formed hydrates then help the fluid flow upwards as they act as a lift mechanism (in the case of positively buoyant hydrate forming compounds) in the return leg, reducing the load on the circulation pump.

Hydrates start dissociating in the upper section of the loop due to a reduction in the hydrostatic pressure and a possible increase in the surrounding temperature. The dissociation of hydrates will result in a reduction in the system temperature, as hydrate dissociation is an endothermic process.

The heat required for hydrate dissociation could be provided by blowing air. This will result in a reduction in the outlet air temperature, which could be used for air conditioning purposes. On the other hand the reduction in the air temperature will result in a reduction in the equilibrium water content in the air. Therefore, the extra water will condense out of the inlet air, resulting in the production of fresh water.

The transference of energy from the air to the dissociating hydrates results in very low air temperatures, the condensation of water vapour, and consequently the production of fresh water.

A pump can be used to regulate the flow rate with respect to the degree of subcooling and the induction time to ensure that hydrates form at the correct section of the loop. However, it is expected that the power input to any pump should be small, where a heat pump with vertical downward and return legs is used, due to hydrate positive buoyancy in the upward leg and cold water negative (and high density after hydrate dissociation) buoyancy in the downward leg.

It is also possible to use a combination of pump and pressure reducing valves to reduce the length of the loop. In this embodiment, the pump is used to pressurise the system and form hydrates (hence releasing heat) and the pressure reducing valves (restrictions) are used to reduce the pressure and dissociate gas hydrates (hence absorbing heat). Various other techniques could be used for pressure increase including roller mechanisms to compress the piping containing the hydrate forming mixture.

Additives can be used to promote hydrate formation and prevent of hydrate blockage within the system. These additives consists of Anti-Agglomerates and compounds that promote the kinetics of hydrate formation by various means, including increasing the hydrate forming compounds solubility, shifting the hydrate stability zone to the right and providing seeds and nucleation sites for gas hydrate formation. For a closed system/loop some Anti-Agglomerates (for example some of the commercial ones mentioned above) may be suitable for the above system because they promote hydrate formation (surfactant effect) and prevent blockage.

The following paper discusses the effect of various types of additives on promoting the hydrate formation rates of natural gas hydrate and its storage capacity, and is of relevance here: “Effect of additives on formation of natural gas hydrate”, C. S. Zhang, S. S. Fan, D. Q. Liang, K. H. Guo. FUEL 83 (16): 2115-2121 NOV 2004.

### PREFERRED EMBODIMENTS OF THE CURRENT INVENTION

In a preferred embodiment of the present invention, according to the first aspect, water (with or without liquid hydrocarbon) is deliberately introduced into a gas, or oil (i.e., above bubble point) or an oil-gas (two phase) pipeline within which the formation of hydrates can be a problem in order to convert most or all the gas phase within the pipeline into hydrates, but where hydrate agglomeration and pipeline blockage is prevented by the use of anti-agglomerates (if necessary), in order to transport the gas phase as hydrates in the form of slurry in the pipeline and in order to increase system capacity and efficiency. The added water could contain additives to promote hydrate formation and/or modify their crystallisation characteristics. AAs could be added to the water phase before or after injection into the pipeline. In the case of a circulating loop, AAs could be added upstream to make up for the amount lost during hydrate and carrier fluid separation.

This embodiment could potentially increase pipeline capacity, as a unit volume of hydrates can accommodate up to 175 units by volume of gas at standard conditions (being roughly equivalent to 2250 psia pressure), as well as reducing pipeline operating pressure, and hence cost of construction and operation of the pipeline. Also as there is no need for pipeline insulation and/or heating, the cost of the pipeline reduces significantly. It is also possible to recover and regenerate all or part of the AAs from the fluids contained within the pipeline downstream and recycle them into the system by injection upstream which, in addition to the recycling of other inhibitors such as scale and corrosion, reduces the chemical costs and alleviates potential environmental concerns. As explained before, the hydrates could be separated by various means, which are not the objective of this invention, including introducing the slurry into a separator and above a sieve tray. The solid hydrates will remain at the top of the tray whereas water containing AAs and other additives will pass through the tray and will be accommodated at the base of the separator. Alternatively, the density difference between hydrates and water could be used for their separation. Hydrates formed from natural gases are generally lighter than water and will accumulate at the top of the separator.

Sea water and/or produced water can be used for the hydrate formation and conversion of the gas phase into hydrates (obviously after taking care of potential scale problems if mixing sea water and formation water), as well as being the carrying fluid. Seawater is generally readily available in an offshore environment. There is no harm in using salt water as carrying fluid, and there is no need to have expensive distilled water even in the case of natural gas systems where the water in the pipeline is generally condensed water. In the case of reservoir formation water, it is already saline water. It is important to ensure the salinity of the free water-rich phase should be below salting-out point (saturation), as well as taking care of scale problems if mixing seawater and formation water. This factor should be accounted for when deciding on the hydrate/free-water ratio in the hydrate slurry. Clearly, it is not desirable to form solid salt crystals. This ratio will depend on the salinity of the produced water, in that the higher the formation water salinity, the lower the maximum hydrate content in the hydrate slurry (should be) in order to avoid salt crystallisation.

Elimination of the gas phase can help flow dynamics significantly and reduce pressure drop across the pipeline. By eliminating (or significantly reducing the gas phase) the system gets nearer to homogenous flow, reducing flow segregation and slugging, improving pressure recovery in downstream flow, and reducing hydrostatic, frictional and acceleration pressure drop components. In addition to flow regime, which is discussed later, the potential benefits will depend on pipeline topography. The higher the number of ups and downs in the pipeline, the higher the benefit of homogenous flow (i.e., pressure recovery in downstream flow). As for frictional pressure drop, there are two possibilities. Firstly, the system does not have much of liquid hydrocarbon phase, i.e., it consists of mainly gas and condensed water. In this case by converting all (or most of the gas into hydrates) a water/hydrate (or water/liquid hydrocarbon/hydrate, or hydrate/liquid hydrocarbon, if liquid hydrocarbon is added to the system) slurry is produced. Considering the densities of water, liquid hydrocarbon and hydrates (which are very close to each other), the system approaches to a homogenous system, reducing the risk of fluid/solid segregation, slugging and hold up. Secondly, when the system does have significant quantities of liquid hydrocarbon, by converting all/most of the gas phase into hydrates and forming hydrates into an oil emulsion or hydrate in water slurry and liquid hydrocarbon phase, again the density difference between the various phases reduces, reducing the risk of fluid segregation and hold up. Another aspect of this embodiment concerns the practical elimination of kinetic energy pressure drop due to the formation of a near incompressible fluid (in comparison to a gas phase which is highly compressible).

Another potential benefit comes from a reduction in overall system pressure, due to lower flow velocities and pressure recovery, allowing for the use of pipes with lower wall thickness. Furthermore, as hydrate formation is an exothermic process, the heat generated during hydrate formation can be used to maintain the system temperature, particularly reducing the risk associated with wax formation and also reducing/eliminating the need for pipeline insulation, reducing the cost of the pipeline as well as installation processes. Furthermore, the risk of wax deposition on the pipe wall is further reduced by the mechanical action of flowing solid hydrate particles, as they remove any solid deposits from the pipe wall, preventing any reduction in the effective pipeline diameter and increase in its surface roughness due to solid deposition. The presence of hydrate crystals could also help in the dispersion of wax particles, reducing the risks associated with wax blockage. It is known that an increase in pipeline internal surface generally result in an increase in system pressure drop.

In an embodiment according to the second aspect of the invention a ring pipeline has circulating water (with or without liquid hydrocarbon and additives) acting as a carrier fluid. In both of the above embodiments the gas could be stored and transported as hydrates (dry, hydrates in oil slurry or hydrates in water slurry) as suggested by other investigators (for example Gudmundsson et al. [“Hydrate Technology for Capturing Stranded Gas” Ann NY Acad Sci. 2000; 912: 403-410.]) with or without dewatering/drying processes. However, as a result of this invention, there would be significant savings in the energy required in the necessary cooling systems in the hydrate formation reactor suggested by the above investigators, as hydrates are formed in the subsea pipelines using the cold subsea environment.

Alternatively, it is possible to recover the gas by dissociating the hydrates by depressurisation and/or heating. The heat source could be water or air. In the case of using air, the outlet air would be much cooler than the inlet air which could be used for air conditioning purposes and/or producing fresh condensed water.

Within the third aspect of the invention an embodiment uses a ring pipeline in conjunction with water (with or without oil and other chemicals) as carrier fluid, but hydrate formation is actively prevented by use of hydrate inhibitor compounds. The degree of subcooling and the level of hydrate inhibitors (thermodynamic or kinetic) can be adjusted, minimised or eliminated by controlling the inhibitor concentration in the circulating carrier fluid, as thermodynamic inhibitors are excluded from hydrate structure, and as hydrate formation results in an increase in inhibitor concentration in free water. Salt concentration should be below salting-out (saturation) concentration to prevent salt deposition.

In embodiments of the fourth aspect of the current invention water and hydrate forming compound(s) are circulated in a closed loop to establish a novel hydrate (heat) pump/cycle utilising the heat generated and/or absorbed during hydrate formation and dissociation of hydrates, respectively. Hydrate formation is an exothermic process while its dissociation needs considerable heat. Hydrate formation can be initiated by increasing (or decreasing for some hydrate forming compounds) pressure and/or reducing temperature. For dissociating hydrates, it is necessary to increase the system temperature and/or reduce (or increase, depending on the type of hydrate forming system) its pressure. Several methods are possible for changing system temperature and/or pressure, including using a pump and/or changes in hydrostatic pressure and/or other means combined with natural or artificial changes in the system temperature.

In one particular embodiment, the system is established in the ocean as a vertical or near vertical closed loop where the rate of circulation could be regulated by a pump. The loop has a downward and an upward leg and two horizontal or inclined segments for assisting hydrate formation and dissociation. Hydrates are formed at the base of the downward leg due to an increase in the system pressure (ideally combined with a decrease in the ambient temperature) releasing heat that could be used for heating purposes. During upward movement the hydrostatic pressure is reduced and the hydrates are dissociated in the surface segment due to a reduction in the system temperature. The reduction in the system temperature could be achieved by using pressure reduction valves or restrictions, reducing the length of the loop. Hydrate dissociation needs heat, hence resulting in a significant reduction in the system temperature which could be used as a refrigeration system for various purposes, including air conditioning and fresh water production, if air is used for providing the necessary heat for hydrate dissociation. The process of cooling air will result in a reduction in its water content, hence extra water will condense which could be used for human consumption, as well as agriculture.

This method simulates nature in a sense that air is transported to low temperature conditions and water is condensed from that air as rain. Simply, it involves the cooling of air which saturated or partially saturated with water vapour in order to condense the water held within.

It worth mentioning that the hydrates (positive buoyant hydrates) formed at the base of the loop will help the fluid flow as they act as a lift mechanism towards areas of lower pressure in the return leg. On the other hand the dissociating hydrates at the upper section of the loop, as a result of reduction in the pipeline pressure, will result in a reduction in the pipeline temperature, as hydrate dissociation is an endothermic process. This could result in an increase in the system density helping downward movement of the fluid. The two processes mentioned above will greatly help the natural circulation, reducing the load on the circulating pump and improving the system economy.

To reduce the reduction in system performance due to formation of a free gas phase during hydrate dissociation, it is necessary to use highly soluble hydrate forming compounds or increase the solubility of the clathrate guest by using additives and/or emulsions, and/or increasing the system pressure. Potential candidates are Tetra HydroFuran (THF) and Tetra-n-Butyl-Ammonium Bromide (TBAB) or other highly water soluble compounds with/without other guest molecules. The ratio between the water and the hydrate forming compounds is adjusted to optimise the hydrate slurry handling (pressure drop) and the overall thermal capacity of the system.

It is important to control hydrate formation in the downward leg of the circulating leg, as immature hydrate formation could result in unwanted upward buoyancy in the case of positive buoyancy compounds (i.e., compounds that their hydrate density is lower than water). Clearly, for negative buoyancy compounds (like CO₂), where their hydrates are heavier than water, the formation of hydrates in the downward leg is helpful for downward movement of the fluid system, but the system will need more energy for upward movement. On the other hand, hydrate dissociation should be controlled in the upward (return) leg to optimise the use of the cooling energy.

Within this embodiment it is possible to adjust the temperature, and through this the rates of hydrate formation (heat release) and hydrate dissociation (cold end) by choosing the type of hydrate former, and also through controlling the system pressure, the vertical and total length of the system.

Hydrate formation is controlled through adjusting the loop pressure to form and dissociate hydrates at specific depths by controlling the rate of fluid circulation, heat removal (e.g., using insulation) and/or type of hydrate forming compounds.

Various additives (for example hydrate promoters, emulsifiers, hydrotropes, anti-agglomerates, surfactants) as well as mechanical means such as mixing can also be added to the system to control the rate of hydrate formation, size of hydrate particles and the system temperature for hydrate dissociation.

The heat required for hydrate dissociation can be supplied by the air (or water) circulating around/through the system (or similar designs for heat exchange purposes), resulting in very low air (or water) temperatures and the condensation of water vapour, and consequently the production of fresh water, as well as air conditioning if desirable.

The necessary heat is supplied to the system by circulating air through natural or forced convection (by fans or blowers) through the system which will result in a reduction in the air temperature and its water content. This water is condensed and removed as fresh water, for example through collectors and pumping.

The cooled air can also be used for air conditioning purposes as the hydrates are in fact working as a heat pump for transporting the cold of the seabed to the surface.

It is also possible to use a secondary water loop for cooling and even freezing water (for water desalination or other purposes). If the ambient air is very dry or it is not possible to extract fresh water, it is possible to use water or a refrigerant to dissociate hydrates. The resulting refrigerant will be cooled which can transfer the cold to another refrigeration system.

Other embodiments of this aspect include methods for supplying heat and energy to subsea facilities, stations, submarines and such like. Here, the heat released as a result of hydrate formation can be used to provide heat to subsea facilities. Again this is achieved through using heat exchangers by passing the cold seabed water over the closed circuit loop. The heat released during hydrate formation will heat the seabed water, resulting in an increase in the outlet water temperature.

### BENEFITS OF THE CURRENT INVENTION

Within the oil industry, the current invention reduces the cost of pipelines by eliminating the need for pipeline insulation or active heating. Furthermore it potentially reduces pipeline operating pressure and increases its capacity.

Within the current invention the costs associated with various inhibitor injection are improved by returning and circulating some of these inhibitors.

The current invention reduces the risks associated with wax formation by controlling the system temperature through exothermic hydrate formation, as well as mechanically removing wax particles from pipeline internal wall by moving solid hydrate particles. Also, the interaction between wax and hydrate particles will result in more dispersed wax precipitation, reducing the risks associated with wax blockage. Furthermore, the removal of wax deposits from pipeline walls reduces their roughness, reducing the frictional pressure drop.

The current invention reduces the risks associated with gas hydrate blockage and the associated costs. It also reduces the risks and the costs associated with pipeline shut-downs and start-up by forming stable hydrate slurries.

The current invention could eliminate the risks associated with slugging and flow instability, reducing/removing the need for slug catchers and the associated CAPEX. It can also reduce the costs associated with preventing corrosion by recycling corrosion inhibitors and in some cases reducing/eliminating the free water.

Some of the existing techniques used by the industry are expensive and risky. The current invention reduces the costs of offshore and deepwater development.

The current invention is economical and involves a degree of purity and flexibility in its range of application.

The current invention helps the environment by reducing and recycling hydrate and other inhibitors.

This innovation could be of particular interest to mature fields where high water cut is a major obstacle in using conventional hydrate prevention techniques.

This innovation could be of particular importance to schemes considering transportation of oil and gas in the form of solid hydrates, as it completely eliminates the hydrate formation stage and reactor and the associated costs.

The heat required for hydrate dissociation could be provided by using air as heating media. This will result in air-conditioning and fresh water production.

The above concept could be used in a hydrate heat pump for heating and/or refrigeration system.

Considerable work has been undertaken on AA and hydrate slurry transportation in water-oil emulsions. The morphology of hydrates formed in a continuous water phase has been investigated using glass micromodels. The use of existing AAs in controlling hydrate blockage in hydrate-water slurry has been investigated using existing kinetic rigs. Typical pressure drop associated with hydrate slurries have been examined. The results show that there is no need for AA for some systems. Furthermore, the results show that the apparent viscosity for hydrate-oil systems could be lower than water-oil systems and this value passes through a minimum as a function of the amount of hydrates in the system.

What is claimed is:

1. A method for transporting a fluid comprising a clathrate forming gas/compound through a transportation system including a pipeline, said method comprising the steps of:
a) subjecting the fluid to clathrate forming temperature and pressure conditions;
b) introducing sufficient of a clathrate forming host to convert substantially all of the gas/compound to said clathrate and to form a flowable slurry unless sufficient clathrate forming host is already present in the fluid; and,
c) conveying the resultant flowable slurry through the transport system to a destination.

2. A method according to claim 1 wherein the clathrate is dissociated at the destination to recover the gas or hydrate forming compounds.

3. A method according to claim 2 wherein the enthalpy of dissociation of the clathrate at the destination is used for cooling.

4. A method according to claim 1 wherein the fluid is a hydrocarbon or hydrocarbon dominated fluid.

5. A method according to claim 1 wherein the clathrate forming host is water.

6. A method according to claim 5 wherein the flowable slurry is a hydrate dispersed in a liquid which is substantially hydrocarbon.

7. A method according to claim 4 wherein the flowable slurry is a hydrate dispersed in a liquid which is substantially water.

8. A method according to claim 1 wherein the method further comprises the step of introducing anti-agglomerate chemicals to the fluid.

9. A method according to claim 8 wherein the anti-agglomerate chemicals are introduced to a concentration of 1-5 vol % of the slurry.

10. A method according to claim 8 wherein the anti-agglomerate chemicals used comprise highly branched quaternised alkyl ammonium or phosphonium compounds, separately or in combination.

11. A method according to claim 8 wherein at least a portion of the anti-agglomerate or other chemicals are recycled for reuse.

12. A method according to claim 1 wherein at least a portion of the clathrate host or other fluids are recycled for reuse.

13. A method according to claim therein the pipeline is a loop.

14. A method according to claim 1 wherein the transport system comprises a ring pipeline, said ring pipeline containing a flow of circulating carrier fluid and connecting at least one source of said fluid to at least one destination.

15. A method according to claim 14 wherein the circulating carrier fluid is substantially water or substantially liquid hydrocarbon.

16. A method according to claim 1 wherein substantially all of the pipeline is at the bottom of the sea.

17. A heat pump wherein the working fluid comprises a clathrate forming host and a clathrate forming guest said host and said guest forming a clathrate and then dissociating back to the host and the guest under the influence of mainly pressure changes.

18. A heat pump according to claim 17 wherein the host is an aqueous fluid.

19. A heat pump according to claim 18 wherein the guest is selected from THF, TBAB, and other hydrate forming compounds.

20. A heat pump as claimed in claim 17 wherein the heat pump comprises two substantially parallel substantially vertical pipes joined in fluid communication at top and bottom to form a loop, wherein the working fluid is pressurised when at a lower portion of the loop relative to the pressure at an upper portion of the loop.

21. A working fluid for a heat pump comprising a clathrate forming host and a clathrate forming guest.

22. A working fluid for a heat pump according to claim 21 wherein the clathrate forming host is an aqueous fluid.

23. A working fluid for a heat pump according to claim 21 wherein the clathrate forming guest is selected from THF, TBAB, and other hydrate forming compounds.

24. A transport system, for a fluid comprising a clathrate forming gas, said system comprising a ring pipeline, said ring pipeline having a circulating carrier fluid comprising clathrate inhibiting compounds.

25. A transport system according to claim 24 wherein the circulating carrier fluid comprises water.

26. A method for transporting a fluid comprising a clathrate forming gas/compound comprising the steps of:
a) inserting said fluid via an inlet into a ring pipeline, said ring pipeline having a circulating carrier fluid comprising clathrate inhibiting compounds; and abstracting said fluid from said circulating carrier fluid at a destination.

27. A method according to claim 26 wherein the circulating carrier fluid comprises water.
